(19) United States
(12) Patent Application Publication
(10) Pub. No.: US 2007/0098647 A1
(43) Pub. Date: May 3, 2007

(54) SKIN CARE PRODUCTS

(75) Inventor: Thomas Neubourg, Werne (DE)

Correspondence Address: DARBY & DARBY P.C., P. O. BOX 5257, NEW YORK, NY, 10150-5257, US

(73) Assignee: Neubourg Skin Care GmbH & Co. KG, Greven (DE)

(21) Appl. No.: 11/535,526

(22) Filed: Sep. 27, 2006

Related U.S. Application Data
(60) Provisional application No. 60/721,170, filed on Sep. 28, 2005.
(60) Provisional application No. 60/785,391, filed on Mar. 23, 2006.

(51) Int. Cl. A61K 8/73; A61K 8/00

(52) U.S. Cl. 424/47; 424/70.13

(57) ABSTRACT

The invention relates to a formulation comprising an aqueous emulsion and a propellant gas for a foam skin cream, wherein the emulsion comprises urea and hyaluronic acid, at least one free fatty acid and at least two emulsifiers, wherein the hyaluronic acid has a molecular weight of at least 1×10⁵ Da.

The invention relates to skin care products in particular for the treatment and care of dry skin conditions and chronic dermatosis. The skin care products are useful for dermatosis comprising atopic dermatitis, the allergic contact eczema, the irritative contact eczema, psoriasis and are in particular useful for the treatment of dry skin and its accompanying symptoms such as inflammations, ulcers, etc., which are associated to diabetes mellitus like the diabetic foot syndrome.

A common skin characteristic of diabetic foot syndrome (DFS) is its dryness accompanied by the disposition to formation of fissures and rhagades as well as the development of hyperkeratosis on pressure-exposed parts.

It is currently assumed that a person suffering from diabetes displays a reduced secretion from the sweat glands on the foot sole (and also partially on the palms) caused by the autonomous neuropathy. The incomplete liquid film promotes direct and indirect drying of the skin by reduction of the effects of evaporation cooling. The correlation can be shown as follows. The autonomous neuropathy leads on the one hand to reduced sweat production and on the other hand to the opening of arteriovenous shunts (autosympathectomy), whereby the reduced sweat production leads to an incomplete liquid film and to a reduction of the evaporation cooling. This leads, together with the opening of the arteriovenous shunts to hyperthermia, which combined with incomplete liquid film leads to the drying of the skin on the foot soles and the palms of the hands. It has been shown that the dryness of the diabetic's skin on the foot sole and on the palms is primarily based on a lack of moisture not on a lack of fat.

It is an object of the present invention to provide skin care products that are suitable for the treatment of diabetic foot syndrome as well as for the treatment of dermatosis, such as atopic dermatitis, the allergic contact eczema, the irritative contact eczema, psoriasis and in particular for the treatment of dry skin and its accompanying symptoms such as inflammations, ulcers etc. that may arise with diabetes mellitus, the diabetic foot syndrome as well as the prophylaxes of these conditions.

The object is solved by the formulations and skin care sets, which are described in detail in the claims and in the following description.

In one aspect the present invention relates to formulations for a foam skin cream, comprising an aqueous emulsion and a propellant gas characterized in that the emulsion comprises urea and hyaluronic acid, at least a free fatty acid and at least two emulsifiers, whereas the hyaluronic acid has a molecular weight of at least 1×10⁵ Da.

In another aspect the invention is directed to formulations for a foam skin cream, which comprise an aqueous emulsion and a propellant gas, characterized in that the emulsion comprises urea and a carbohydrate-blend, which is obtained from naturally available carbohydrates, at least one free fatty acid and at least two emulsifiers.

In still another aspect the invention relates to formulations for a foam skin cream, comprising an aqueous emulsion and a propellant gas, characterized in that the emulsion comprises at least one free fatty acid and at least two emulsifiers and the pH is adjusted to 5.5 to 8 with potassium hydroxide.

The invention is directed in a further aspect to day and night skin care sets comprising a day skin care composition and a night skin care composition, whereas the day skin care composition is a foam skin cream comprising at least one free fatty acid and at least two emulsifiers, whereas the night skin care composition comprises an additional agent or a higher weight-percentage of an agent or another agent as the day skin care composition, whereas the agent is selected from the group consisting of lipids and hydrated derivatives or mixtures thereof.

In one aspect the invention relates to the use of a formulation or a day- and night care set according to the invention in the treatment of dermatosis, as well as methods for treatment of dermatosis, in which a formulation according to the present invention is applied to the skin area to be treated. Further, in another aspect the invention relates to a method for treatment of dermatosis, in which, on getting up, the day skin care composition for day care and before going to bed the night care skin composition for night care, according to the present invention, is administered on the skin to be treated. Dermatosis are for example, dry skin conditions, chronic dermatosis, atopic dermatitis, allergic contact eczema, irritative contact eczema, psoriasis and the diabetic foot syndrome.

In the following, the specific aspects of the invention are described in detail. The specific aspects of the invention can be used alone or combined advantageously. The features described individually can therefore be combined with all other features that are described in the present invention.

In one aspect the invention relates to foam creams.

Foam creams are aerosols that are formed with a propellant gas by spraying from a suitable container/dosage form.

Foam cream formulations therefore in general contain an emulsion and a propellant gas. Within the present invention the term “formulation” relates to all ingredients, which are necessary to create a foam cream, i.e. the emulsion and the propellant gas, whilst the term “emulsion” relates to all ingredients other than the propellant gas.

As already mentioned, foams are aerosols and are complex, physical-chemical structures, which require a special balance of the foam-forming components. The characteristic of the foam cream of the present invention is that by application onto the skin a net-like structure, comparable to a membrane, is formed by its stable two-phase polydispersed structure. In particular the fatty acid component forms finely woven, net-like crystals that act like a sieve with a small wetting angle of contact to prevent the contact of water with the skin. On the other hand, the skin between the meshes remains practically unchanged and can therefore perform the unhindered gas exchange with the environment. There is practically no hindrance of insensible persperation, no hyperthermia and no change in the feel due to the automatically predetermined thin application of the foam. These properties allow for an unchanged cutaneous respiration and simultaneous protection function.

Spraying a formulation that contains an aqueous emulsion and a propellant gas from a pressurized container forms the foam skin cream according to the present invention. Suitable propellants are all pharmaceutically acceptable propellants that can be used for the formation of foam creams.

In the meaning of the present invention, the aqueous emulsion contains all components of the formulation except the propellant gas.

According to one aspect the foam cream comprises urea and hyaluronic acid. In particular, the foam cream according to the present invention comprises urea and high molecular hyaluronic acid with a molecular weight M from at least 1×10⁵ Da, at least 5×10⁵ Da, at least 1×10⁶ Da or at least 1.5×10⁶ Da. In one aspect of the present invention hyaluronic acid of about 1.7×10⁶ Da is applied. In the meaning of the present invention, hyaluronic acid means either the free hyaluronic acid or a corresponding pharmaceutically acceptable derivative thereof, in particular a pharmaceutically acceptable salt. However, amounts and size indications always refer to the free hyaluronic acid in the context of the present invention.

The urea affects an increase in water-binding capability of the horn layer inter- as well as intracellular. Its low molecular weight and the resulting strong osmotic properties as well as its polarity and the hygroscopic properties resulting thereof impart the effectiveness of urea. In its native form, urea is a bipolar molecule and on the positive and negative pole, preferably water molecules accumulate by means of hydrogen bonding and therefore impart the water binding capacity of the urea.

Hyaluronic acid is a natural, endogenous polymer made of carbohydrates and is strongly hygroscopic. Due to its physical-chemical structure, hyaluronic acid forms a network of polysaccharides that is capable of binding water and improving the elasticity of the epidermis. In particular, high molecular weight hyaluronic acid is capable of forming such networks and act by its physical properties in the foam cream according to the present invention. In particular it has been shown by the present invention that the hyaluronic acid stabilizes the foam structure and therefore affects the formation of the net structure on the skin and thus the related protection function.

According to this aspect, the cream foams of the present invention supply the skin, in particular especially stressed and dry skin of diabetics with sufficient moisture without clogging the pores and thus without leading to a further reduction of the sweat formation. Furthermore, the cream foams according to the present invention prevent the skin from being overloaded with greasing substances and the cause of an uncomfortable greasy feeling. The cream foams according to the present invention quickly penetrate into the skins surface leaving a pleasant skin feeling without greasiness. These cream foams are particularly suitable for day care products.

The aqueous emulsion comprises urea, hyaluronic acid, at least one free fatty acid and at least two emulsifiers according to this aspect of the invention.

In the following the composition of the emulsion will be described in detail.

According to this embodiment the foam cream comprises urea and high molecular hyaluronic acid. The hyaluronic acid has a molecular weight of at least 1×10⁵ Da or at least 5×10⁵ Da or at least 1×10⁶ Da and in particular of at least 1.5×10⁶ Da. According to one embodiment hyaluronic acid of about 1.7×10⁶ Da is used.

According to one embodiment the emulsion comprises from 3-30 wt-% urea or 4-30 wt-% urea or 5-20 wt-% urea or 5-17 wt-% urea. Preferred embodiments contain more than 5 wt-% urea such as 5.5 to 25 wt-% or 5.5, 11 or 16.5 wt-% urea.

In one embodiment the emulsion further comprises 0.01-5 wt-% hyaluronic acid (based on the weight of the free acid) or 0.01-3 wt-% or 0.1 to 1 wt-% hyaluronic acid (based on the weight of the free acid). Preferred embodiments contain less than 1 wt-% hyaluronic acid or less than 0.5 wt-% hyaluronic acid like about 0.01-0.9 wt-%, 0.01-0.5 wt-% or 0.2 wt-% hyaluronic acid.

In another aspect of the present invention the foam cream contains urea and a carbohydrate blend, which is obtained from natural available carbohydrates. According to one embodiment of the present invention, the carbohydrate blend corresponds in its composition to the carbohydrates contained in human skin. This carbohydrate blend is for example applied in form of an aqueous solution of 50-55 wt-% dry residue which is for example available under the trade name Pentavitin® from the company Pentapharm. This is a product that is obtained by careful isomerisation of D-glucose obtained from plants. Such carbohydrate blends are suitable to bind water in the skin over a long period and effectively. Such carbohydrate blends are also called moisture magnets. These contribute in the formulations according to the invention in that the moisture, which is for example donated by the urea and/or hyaluronic acid, is bonded in the skin.

According to this aspect the cream foams according to the present invention supply the skin, in particular the stressed and dry skin of diabetics with sufficient moisture without clogging the pores and are also able to bind the moisture over a long period. Again, the cream foams according to the present invention prevent an overload of the skin with greasing substances and therefore prevent an unpleasant greasy feel. The cream foams penetrate quickly into the skin and leave a pleasant skin feel without greasiness. These cream foam formulations are also well suited for day- and night care.

In the following, the composition of the emulsion is described in detail.

According to this embodiment, the foam cream comprises urea and the carbohydrate blend. According to one embodiment the carbohydrate blend is obtained from naturally available carbohydrates and corresponds preferably in its composition to the carbohydrates contained in human skin. The carbohydrate blend can be applied in form of an aqueous solution with 50-55 wt-% dry residue, which is available under the trade name Pentavitin®.

According to one embodiment the emulsion comprises 3-30 wt-% urea or 4-30 wt-% urea or 5-20 wt-% urea or 5-17 wt-% urea. Preferred embodiments contain more than 5 wt-% urea, for example 5.5-25 wt-% urea, like about 5.5, 11 or 16.5 wt-% urea.

In one embodiment the emulsion further comprises 0.01-5 wt-% of the aqueous solution of the carbohydrate blend or 0.01-3 wt-% of this aqueous solution and preferably about 0.6 wt-% or 1.25 wt-% of this aqueous solution.

According to another embodiment of the invention the emulsion comprises urea, high molecular hyaluronic acid and the carbohydrate blend in forms and amounts, which are described above. According to one embodiment the emulsion comprises 5-17 wt-% urea, or more than 5 wt-% urea, like about 5.5 to 25 wt-% urea, 0.01-1 wt-% hyaluronic acid or less than 1 wt-% hyaluronic acid, like about 0.001-0.9 wt-% or 0.01-0.9 wt-% hyaluronic acid and 0.01-2 wt-% of the carbohydrate blend. According to a preferred embodiment the emulsion contains 5-17 wt-% urea, about 0.2 wt-% hyaluronic acid and about 0.6 wt-% or about 1.25 wt-% of the carbohydrate blend in form of an aqueous solution.

A further aspect of the invention is directed to the stability of the foam (stability of the foam structure), in particular the storage stability of the formulation in the pressurized container with respect to the resulting foam stability. As mentioned above, foams are aerosols and are complex physical structures, which in particular by their stable two-phase polydisperse structure form a net structure when applied to the skin that contributes to the advantages of the foam formulations. In case the foam structure collapses too quickly, this net structure cannot be formed. According to one aspect of the present invention, the stability of the foam structure, in particular the foam stability upon storage is improved.

It has been shown that the stability of the foam or the foam structure can be influenced advantageously by the selection of the base applied for adjusting the pH-value. It has been shown that among the bases like aminomethylpropanol (AMP), sodium hydroxide (NaOH) and potassium hydroxide (KOH), the application of potassium hydroxide leads to an improvement in foam stability, in particular upon storage between 10 and 40° C. This effect has been shown by the application of a base emulsion comprising free fatty acid and at least two emulsifiers as well as a combination of various active agents. According to this aspect of the invention, the foam formulation comprises an aqueous emulsion and a propellant gas, whereas the emulsion comprises at least a free fatty acid and at least two emulsifiers and the pH is adjusted by means of potassium hydroxide to 5.5-8.

In the context of the present invention the term “free fatty acid” means a free fatty acid, a blend of free fatty acids as well as a partially saponified fatty acid or a blend of partially saponified fatty acids, respectively.

It has further been established that the addition of hyaluronic acid has a stabilizing effect on the foam structure. In particular it has been found that the addition of 0.2 wt-% hyaluronic acid has an especially stabilizing effect. According to this aspect of the invention, the emulsion comprises at least a free fatty acid, at least two emulsifiers and about 0.2 wt-% hyaluronic acid and the pH is adjusted by means of potassium hydroxide to 5.5 to 8. According to one aspect of the present invention the emulsion is adjusted to a pH of 6.5 to 7.5 with potassium hydroxide.

The described gain in stability by the adjustment of the pH by means of potassium hydroxide as well as the improvement of the stability by the addition of hyaluronic acid can be easily combined with different aspects of the invention, that are described herein.

In the following the emulsion foundation of the base emulsion is described, which can be easily combined with all aspects of the invention, such as for example the ingredients/active agents, urea, hyaluronic acid and carbohydrate blends as well as the pH adjustment by potassium hydroxide as described above, as well as the day- and night skin care sets described below.

The emulsion is preferably an oil-in-water emulsion (O/W-emulsion), which foams upon exiting the valve of the pressurized container by the expansion of the propellant gas, dissolved in the inner oil phase.

Such O/W-emulsions consist of an lipophilic phase I and a hydrophilic phase II, that upon addition of emulsifiers under suitable mixing conditions form emulsions, whereas the outer phase is the water phase/hydrophilic phase and the inner phase is the oil phase/lipophilic phase. For this, according to the present invention the lipophilic/oil phase also called fatty phase is melted or heated, as well as suitable emulsifiers (one emulsifier or a blend of many different emulsifiers) are added and are metered to the hydrophilic phase/water phase, which is also heated, which also contains emulsifier (one emulsifier or a blend of many different emulsifiers) with stirring. It is also possible to meter the hydrophilic phase to the lipophilic phase. The homogenous blend is subsequently cooled according to the present invention and the pH is adjusted, if necessary. The cooled mixture/emulsion obtained is then filled in a suitable dosage form with the addition of the propellant gas.

The emulsions according to the present invention contain free fatty acids and at least two emulsifiers. C₁₂- to C₂₂-fatty acids are preferred, in particular natural C₁₂- to C₂₀-fatty acids, for example stearic acid, palmitic acid and myristic acid or mixtures thereof. Stearic acid is particularly preferred. Stearic acid means in the context of the present invention either substantially pure stearic acid or a blend of higher fatty acids like stearic acid and palmitic acid. The fatty acids are components of the lipophilic phase/fatty phase. The emulsions according to the present invention contain preferably 1 to 10 wt-% fatty acids, more preferably 4 to 7 wt-% fatty acids and even more preferably 4.5 to 6 wt-% fatty acids.

According to one embodiment of the invention, the emulsion further comprises unsaturated and/or polyunsaturated fatty acids such as omega-6-fatty acids, like for example known from evening primrose oil and borage oil. Synthetic omega-6-fatty acids or also those that can be obtained from plants may be used. According to a further aspect of the present invention, the emulsion contains evening primrose oil.

The emulsion further comprises at least two different emulsifiers, that are selected on the one hand from the group consisting of non-ionic emulsifiers, in particular emulsifiers based on fatty alcohol base and on the basis of partial esters of fatty acids such as cetearyl alcohol or glyceryl stearate and on the other hand are selected from the group of anionic emulsifiers, in particular alkyl sarcosinates such as lauryl, lauroyl or cetyl sarcosinate. Preferably the non-ionic emulsifiers are initially added to the fatty phase and the anionic emulsifiers are added to the water phase during preparation. The emulsion according to the invention contains preferably from 4 to 15 wt-% of these emulsifiers. Preferably the emulsion contains at least three emulsifiers, whereas two emulsifiers are selected from the group of non-ionic emulsifiers and one emulsifier is selected from the group of ionic emulsifiers. The combination of cetearyl alcohol, glyceryl stearate and lauryl sarcosinate is most preferred. In these combinations preferably 1 to 3 wt-% glyceryl stearate, 3 to 6 wt-% cetearyl alcohol and 1 to 3 wt-% lauryl sarcosinate are used. Further suitable emulsifiers or stabilizing ingredients are cetearyl glycoside, hydroxyl ethyl acrylate, sodium acryloyl dimethyltaurat copolymer, polysorbate 60, Squalan, and mixtures thereof and the like.

In a preferred embodiment of the invention the emulsion comprises further at least an additional emulsifier, which is according to the present invention referred to as a co-emulsifier. This co-emulsifier is selected from the group of lipophilic emulsifiers, in particular from the group selected from triceteareth-4-phosphate, Olet-3-phosphate as well as further lipophilic emulsifiers on the basis of lower ethoxylated fatty alcohols. The co-emulsifier is preferably initially added to the fatty phase during preparation. Preferably 0.4 to 2.5 wt-% are used. Preferably triceteareth-4-phosphate is used in amounts of 0.4 to 2.5 wt-%.

According to a further preferred embodiment the emulsion further comprises refatting substances. These refatting substances are preferably selected from the group consisting of decyl oleate, isohexadecane, stearic acid glycol ester, coconut fatty acid ethanolamide, corn oil, peanut oil, almond oil, sesame oil, olive oil, jojoba oil, soya bean oil, wool wax alcohols, paraffin, medium-chain triglycerides, oleic acid oleyl esters, white petrolatum, macrogol-glycerol hydroxystearate, hydrogenated castor oil, castor oil from Ricinus communis, avocado oil, wheat germ oil, evening primrose oil, shea butter, palmitic acid isopropyl ester, cetyl palmitate, myristic acid myristyl ester and octyldodecanol. The emulsions according to the invention contain 0.5 to 6 wt-% refatting substances, preferably 0.5 to 2 wt-% refatting substances. Further oil components are C₁₂-C₁₃-alkyl lactates and C₁₂-C₁₅-alkyl benzoates. Too much fatting substance should be avoided according to some aspects of the invention. For day care, low fat products are suitable and for night care fatty products are suitable. According to one embodiment the emulsion is free of paraffin.

The emulsions according to the invention can further contain moisturizers, preservatives, soothing substances, skin care agents and skin active vitamins as well as silicone-containing substances such as dimethicone.

Propylene glycol and/or polyhydric alcohols, in particular glycerine, may be used as further moisturizers. Moisturizers can be added in an amount of from 1-10 wt-%, preferably from 2.5-8.5 wt-%. Preferably glycerine and propylene glycol are added.

As preservatives in particular substances like parabene, methyl dibromo glutaronitrile and/or phenoxy ethanol are suitable. Preservatives may be used in amount from 0.01-1 wt-%. It is preferred not to add any preservatives.

Suitable soothing substances, skin care agents and skin active vitamins are chamomile, calendula, hamamelis and tea tree oil extracts, portulaca, allantoin, panthenol and the vitamins A, E and F. These ingredients can be used in amounts from 0.01-2.0% or 0.05-1 wt-%. It is preferred to use allantoin.

Portulaca is used as a soothing substance according to one aspect of the invention. Portulaca can be used in amounts from 0.01-2 wt-%, preferably about 0.1 wt-%.

The formulations according to the invention further have a pH-value that is acceptable for the skin, like about from 7.0 to 8.0, 5.5 to 8 or 6.5 to 7.5. 2-Amino-2-methyl-1-propanol (AMP) can also be applied. According to one aspect potassium hydroxide is used as described above.

A formulation according to the invention contains an emulsion, which comprises from 1-10 wt-% fatty acids, 4-15 wt-% emulsifiers, 0.4-2.5 wt-% co-emulsifiers, 1-10 wt-% moisturiser, 0.05-1 wt-% skin care agent and 0.5-6 wt-% refatting substances. A further formulation according to the invention contains an emulsion, which comprises from 3-7 wt-% stearic acid, 4-10 wt-% emulsifiers, 0.4 to 2 wt-% co-emulsifiers, 2.5-8.5 wt-% moisturizer, 0.5-2 wt-% refatting substances and 0.05-1 wt-% skin care agents. In this emulsion glyceryl stearate and cetaryl alcohol and sodium lauryl sarcosinate are preferably used as emulsifiers and triceteareth-4-phosphate is used as co-emulsifier. Decyl oleate and octyldodecanol are preferably used as refatting substances and glycerine is preferably used as moisturizer and allantoin is preferably used as a skin care agent. To the emulsion, for example urea, hyaluronic acid and Pentavitin can be added, as described above.

Water is added for adjustment to 100%.

The formulations according to the invention are prepared as follows:
preparing a Phase I by melting at 70° C.-80° C., preferably at 75° C. a mixture comprising at least one fatty acid, at least one non-ionic emulsifier and at least one co-emulsifier, and optionally the refatting substances,
followed by metering to Phase II, which is tempered to 70°-80° C., preferably to 75° C., comprising water, urea, hyaluronic acid and at least one anionic emulsifier as well as optionally moisturizers and skin care agents with stirring to produce a homogenous mixture/emulsion of Phase I and Phase II,
cooling of the mixture/emulsion to a temperature from room temperature to 40° C., preferably 30° C. to 40° C. while stirring and
if necessary adjustment of the pH, preferably to a pH of 5.5-8.
Filling of the resulting mixture/emulsion in a suitable dosage form under addition of a propellant gas.

According to a further aspect the invention relates to a day- and night skin care set, comprising a day skin care composition and a night skin care composition, the day skin care composition is a foam skin cream, comprising at least one free fatty acid and at least two emulsifiers and the night skin care composition comprises an additional active agent or a higher weight percentage of active agent or different active agents as the day skin care composition, whereas the active agent is selected from the group consisting of lipid substances and hydrogenated substances or mixtures thereof.

Day and night differ with respect to the skin care by different requirements with respect to the ease of application and the care requirements. During the day it is important that the skin care is less greasy and interferes only minimally with the day's activities. Further, an improved protection function is desired. During the night the ease of application is less important as well as the protective function and more nourishing and usually greasier substances can be used. The nighttime can therefore be effectively used for the care and regeneration while during the day the protective function and the ease of use are more important. Therefore a combination in form of a day- and night skin care set, comprising a special day skin care composition which is suited to daytime requirements and a night skin care composition which is especially formulated to satisfy night time needs. A foam skin cream is used, as a day skin care composition according to the invention, which can, for example, be one of the formulations described above. In particular the foam formulations provide an improved application comfort and a good protection function, which correspond to the day application. A foam skin cream can also be used as a night skin care composition, while here also more greasy ointments and lotions can be used. According to one embodiment of the invention, the active agents contained such as lipid substances and hydrogenated substances of the day skin care composition and the night skin care composition are balanced. On the one hand, both skin care compositions may contain the same active agent or the same combination of active agents whereas the night skin care composition, for example, contains comparably higher contents of the active agents than the day skin care composition. Further, the night skin care composition can contain additional active agents or different active agents. Here any combination of active agents come into question, in particular the combination of active agents as described above, for example urea, hyaluronic acid and carbohydrate blend. These are hydrated substances, which donate moisture and store it in the skin. Further lipid substances can be used, for example fatty acids, inter alia, unsaturated fatty acids as well as any refatting substances. According to one embodiment of the invention, evening primrose oil, which has a high content of unsaturated fatty acids, is used as a lipid substance.

According to a further aspect of the invention the night skin care composition described above may possibly be used as a day skin care composition and the day skin care composition may be used as night skin care composition.

According to one aspect of the invention, the day skin care composition is a formulation comprising urea and hyaluronic acid as described above and the night skin care composition is a formulation containing urea, hyaluronic acid and the carbohydrate blend. In particular, for the day skin foam cream a formulation comprising 11% urea, 0.2% hyaluronic acid may be used and adjusted to a pH of about 7 by means of addition of KOH as a base. This formulation will be combined with a night skin care comprising a formulation with 11 wt-% urea, 0.2 wt-% hyaluronic acid as well as 0.6 or 1.25 wt-% of carbohydrate blend in form of Pentavitin® and a pH is adjusted to about 7.

According to a further aspect the day skin care is a formulation containing preferably 5 to 17% urea or 5 to 15% urea and the night skin care is a formulation containing a carbohydrate blend and optionally hyaluronic acid as described in the present specification or vice versa.

According to another aspect the day skin care is a formulation containing 16.5% urea and 1% evening primrose oil, whereas the formulation can also be adjusted to a pH of about 7 by means of KOH. As night skin care, for example, an ointment may be used which is described in the following exemplary compositions.

The day and night skin care set according to the present invention and as described above comprise a day skin care composition and a night skin care composition which are suitable for many applications, for example, for the treatment and care of dry skin conditions and chronic dermatosis. But also stressed skin areas, such as hands and feet of athletes may be treated and (further) skin damage may be prevented.

| Ingredient | Trade name | weight-%. |
|---|---|---|
| Urea | | 15 |
| Glycerine | | 6 |
| Glyceryl Stearate SE | (Tegin Pellets) | 8 |
| Isopropyl Palmitate | (IPP) | 3 |
| Octyldodecanol | (Eutanol G) | 3 |
| Persea Gratissima | (Avocado oil) | 2.5 |
| Glyceryl Stearate | (Tegin 4100 Pellets) | 3 |
| Cetyl Alcohol | (Lanette 16) | 2.5 |
| Cyclomethicone | (Belsil CM 040) | 0.5 |
| Dimethicone | (Belsil DM 350) | 0.5 |
| Myristyl Myristate | (Cetiol MM) | 0.3 |
| Alga extract | (Vegetol Algues) | 0.3 |
| Citronelle Methylcrotonate | (Sinodor) | 0.3 |
| Allantoin | | 0.2 |
| BHT | | 0.15 |
| Xanthan Gum | (Kezan ST) | 0.25 |
| Ethyl paraben | (Dekaben MEP) | 0.6 |
| Methyl paraben | | |
| Phenoxyethanol | | |
| Propyl paraben | | |
| Perfume | | 0.5 |
| Water | | 53.4 |

### EXAMPLES

### Example 1

The foam cream is prepared in a heatable and coolable closed apparatus which is equipped with an auto-discharging homogenizer and a heatable metering funnel.

The preparation of Phase I is performed in a heatable metering funnel by melting a mixture comprising 2 wt-% of glyceryl stearate, 4 wt-% cetaryl alcohol, 5 wt-% stearic acid, 1 wt-% paraffin, 1 wt-% triceteareth-4-phosphate at 75° C. This phase is metered with stirring to a Phase II already present in a heatable and coolable closed apparatus which comprises a auto-discharging homogenizer. This phase consists of an aqueous mixture comprising 2.5 wt-% propylene glycol, 2.5 wt-% glycerine, 2 wt-% sodium lauryl sarcosinate as well as 0.3 wt-% allantoin and 10 wt-% urea as well as 0.1 wt-% hylauronic acid having a molecular weight of at least 1.5×10⁶ Da. The quantity of water amounts to 69.6 wt-%. A homogenous blend of Phases I and II is provided.

The metering of Phase I is performed at a temperature of 75° C. Both phases are combined with constant stirring at a medium stirring speed, and a uniform homogenising has to be assured. The temperature is maintained for between 5 and 20 minutes at 75° C. The resulting mixture/emulsion is cooled to a temperature of between 30° and 40° C. with constant stirring.

After the temperature of 40° C. has been reached, further substances may be added. In doing this, the pH-value can also be adjusted between 5.5 and 8 or 6.5 and 7.5. Therefore, 2-amino-2-methyl-1-propanol or KOH is used, while KOH is preferred. Stirring is continued for a sufficient time period until the pH-value has been stabilized and then the filling into suitable storage containers or corresponding spraying containers is performed. Thereby 91 wt-% emulsion together with 9 wt-% butane/propane are combined.

Further illustrative foam cream compositions that may be produced according to the method of Example 1 are described in the following Examples 2 to 7.

### Example 2

5.5% urea and 0.2% hyaluronic acid

| Ingredient | Trade name | g/kg | wt.-% |
|---|---|---|---|
| Decyl Oleate | (Cetiol V) | 50 | 5 |
| Octyldodecanol | (Eutanol G) | 50 | 5 |
| Dimethicone | (Dow Corning | 2 | 0.2 |
| | Fluid 200/350 cst) | | |
| Glyceryl Stearate | (Cutina MD) | 20 | 2 |
| Cetearyl Alcohol | (Lanette O) | 40 | 4 |
| Stearic acid | (Edenor C 18 98/100) | 50 | 5 |
| Hyaluronic acid | | 2 | 0.2 |
| high-molecular | | | |
| weight | | | |
| Triceteareth-4- | (Hostaphat KW 340 D) | 10 | 1 |
| phosphate | | | |
| Water demin. | | 649.4 | 64.94 |
| Urea | | 55 | 5.5 |
| (cosmetic quality) | | | |
| Propylene glycol 1,2 | | 25 | 2.5 |
| Glycerine 86% | | 25 | 2.5 |
| Sodium Lauroyl | (Protelan LS 9011) | 20 | 2 |
| Sarcosinate | | | |
| Allantoin | | 1 | 0.1 |
| KOH | | 0.6 | 0.06 |

| | | |
|---|---|---|
| Total | 1000 | 100 |

### Example 3

11% urea und 0.2% hyaluronic acid

| Ingredient | (Trade name) | g/kg | wt.-% |
|---|---|---|---|
| Decyl Oleate | (Cetiol V) | 75 | 7.5 |
| Octyldodecanol | (Eutanol G) | 75 | 7.5 |
| Dimethicone | (Dow Corning | 2 | 0.2 |
| | Fluid 200/350 cst) | | |
| Glyceryl Stearate | (Cutina MD) | 20 | 2 |
| Cetearyl Alcohol | (Lanette O) | 40 | 4 |
| Stearic acid | (Edenor C 18 98/100) | 50 | 5 |
| Hyaluronic acid | | 2 | 0.2 |
| high-molecular | | | |
| Triceteareth-4- | (Hostaphat KW 340 D) | 10 | 1 |
| phosphate | | | |
| Water demin. | | 544.5 | 54.45 |
| Urea | | 110 | 11 |
| (cosmetic quality) | | | |
| Propylene glycol 1,2 | | 25 | 2.5 |
| Glycerine 86% | | 25 | 2.5 |
| Sodium Lauroyl | (Protelan LS 9011) | 20 | 2 |
| Sarcosinate | | | |
| Allantoin powder | | 1 | 1 |
| KOH | | 0.5 | 0.5 |

| | | |
|---|---|---|
| Total | 1000 | 100 |

### Example 4

11% urea and 0.6% Pentavitin

| Ingredient | Trade name | g/kg | wt.-% |
|---|---|---|---|
| Decyl Oleat | (Cetiol V) | 75 | 7.5 |
| Octyldodecanol | (Eutanol G) | 75 | 7.5 |
| Dimethicone | (Dow Corning | 2 | 0.2 |
| | Fluid 200/350 cst) | | |
| Glyceryl Stearate | (Cutina MD) | 20 | 2 |
| Cetearyl Alcohol | (Lanette O) | 40 | 4 |
| Stearic acid | (Edenor C 18 98/100) | 50 | 5 |
| Carbohydrate blend | (Pentavitin) | 6 | 0.6 |
| Triceteareth-4-phosphate | (Hostaphat KW 340 | 10 | 1 |
| | D) | | |
| Water demin. | | 540.5 | 54.05 |
| Urea | | 110 | 11 |
| (cosmetic quality) | | | |
| Propylene glycol 1,2 | | 25 | 2.5 |
| Glycerine 86% | | 25 | 2.5 |
| Sodium Lauroyl | (Protelan LS 9011) | 20 | 2 |
| Sarcosinate | | | |
| Allantoin | | 1 | 0.1 |
| KOH | | 0.5 | 0.05 |

| | | |
|---|---|---|
| Total | 1000 | 100 |

### Example 5

11% urea und 1.25% Pentavitin

| Ingredient | Trade name | g/kg | wt.-% |
|---|---|---|---|
| Decyl Oleate | (Cetiol V) | 75 | 7.5 |
| Octyldodecanol | (Eutanol G) | 75 | 7.5 |
| Dimethicone | (Dow Corning | 2 | 0.2 |
| | Fluid 200/350 cst) | | |
| Glyceryl Stearate | (Cutina MD) | 20 | 2 |
| Cetearyl Alcohol | (Lanette O) | 40 | 4 |
| Stearic acid | (Edenor C 18 98/100) | 50 | 5 |
| Carbohydrate blend | (Pentavitin) | 12.5 | 1.25 |
| Triceteareth-4- | (Hostaphat KW 340 D) | 10 | 1 |
| phosphate | | | |
| Water demin. | | 534 | 53.4 |
| Urea | | 110 | 11 |
| (cosmetic quality) | | | |
| Propylene glycol 1,2 | | 25 | 2.5 |
| Glycerine 86% | | 25 | 2.5 |
| Sodium Lauroyl | (Protelan LS 9011) | 20 | 2 |
| Sarcosinate | | | |
| Allantoin | | 1 | 0.1 |
| KOH | | 0.5 | 0.05 |

| | | |
|---|---|---|
| Total | 1000 | 100 |

### Example 6

16.5% urea and 0.6% Pentavitin und 0.1% portulaca

| Ingredient | Trade name | g/kg | wt.-% |
|---|---|---|---|
| Decyl Oleate | (Cetiol V) | 75 | 7.5 |
| Octyldodecanol | (Eutanol G) | 75 | 7.5 |
| Dimethicone | (Dow Corning | 2 | 0.2 |
| | Fluid 200/350 cst) | | |
| Glyceryl Stearate | (Cutina MD) | 20 | 2 |
| Cetearyl Alcohol | (Lanette O) | 40 | 4 |
| Stearic acid | (Edenor C 18 98/100) | 50 | 5 |
| Cabohydrate blend | (Pentavitin) | 6 | 0.6 |
| Triceteareth-4-phosphate | (Hostaphat KW 340 D) | 10 | 1 |
| Water demin. | | 484.3 | 48.43 |
| Urea | | 165 | 16.5 |
| (cosmetic quality) | | | |
| Propylene glycol 1,2 | | 25 | 2.5 |
| Glycerine 86% | | 25 | 2.5 |
| Sodium Lauroyl | (Protelan LS 9011) | 20 | 2 |
| Sarcosinate | | | |
| Allantoin | | 1 | 0.1 |
| KOH | | 0.7 | 0.07 |
| Portulaca | | 1 | 0.1 |

| | | |
|---|---|---|
| Total | 1000 | 100 |

### Example 7

16.5% urea and 1.25% Pentavitin und 0.1% portulaca

| Ingredients | Trade name | g/kg | wt.-% |
|---|---|---|---|
| Decyl Oleate | (Cetiol V) | 75 | 7.5 |
| Octyldodecanol | (Eutanol G) | 75 | 7.5 |
| Dimethicone | (Dow Corning | 2 | 0.2 |
| | Fluid 200/350 cst) | | |
| Glyceryl Stearate | (Cutina MD) | 20 | 2 |
| Cetearyl Alcohol | (Lanette O) | 40 | 4 |
| Stearic acid | (Edenor C 18 98/100) | 50 | 5 |
| Carbohydrate blend | Pentavitin | 12.5 | 1.25 |
| Triceteareth-4- | (Hostaphat KW 340 D) | 10 | 1 |
| phosphate | | | |
| Water demin. | | 477.8 | 47.78 |
| Urea | | 165 | 16.5 |
| (cosmetic quality) | | | |
| Propylene glycol 1,2 | | 25 | 2.5 |
| Glycerine 86% | | 25 | 2.5 |
| Sodium Lauroyl | (Protelan LS 9011) | 20 | 2 |
| Sarcosinate | | | |
| Allantoin | | 1 | 0.1 |
| KOH | | 0.7 | 0.07 |
| Portulaca | | 1 | 0.1 |

| | | |
|---|---|---|
| Total | 1000 | 100 |

### Example 8

Day- and night skin care sets

Day care composition: Foam cream formulation with an emulsion comprising 16.5% urea und 1% evening primrose oil

| Ingredient | Trade name | amount/kg | wt.-% |
|---|---|---|---|
| Decyl Oleate | (Cetiol V) | 7 | 7 |
| Octyldodecanol | (Eutanol G) | 7 | 7 |
| Dimethicone | (Dow Corning | 0.2 | 0.2 |
| | Fluid 200/350 cst) | | |
| Glyceryl Stearate | (Cutina MD) | 2 | 2 |
| Cetearyl Alcohol | (Lanette O) | 4 | 4 |
| Stearic acid | (Edenor C 18 98/100) | 5 | 5 |
| Paraffin oil P-615 EL | | 1 | 1 |
| Triceteareth-4-phosphate | (Hostaphat KW 340 D) | 1 | 1 |
| Water demin. | | 47.7 | 47.7 |
| Urea | | 16.5 | 16.5 |
| Monopropylene glycol | | 2.5 | 2.5 |
| 1,2 | | | |
| Glycerine 86% | | 2.5 | 2.5 |
| Sodium Lauroyl | (Protelan LS 9011) | 2 | 2 |
| Sarcosinate | | | |
| Allantoin | | 0.1 | 0.1 |
| Evening primrose oil | | 1 | 1 |
| AMP | | 0.5 | 0.5 |

| | | |
|---|---|---|
| Total | 100 | 100 |

Night care composition in the form of an ointment with the following composition:

| Ingredient | (Trade name) | wt.-% |
|---|---|---|
| Urea | | 15 |
| Glycerine | | 6 |
| Glyceryl Stearate SE | (Tegin Pellets) | 8 |
| Isopropyl Palmitate | (IPP) | 3 |
| Octyldodecanol | (Eutanol G) | 3 |
| Persea Gratissima | (Avocadooil) | 2.5 |
| Glyceryl Stearate | (Tegin 4100 Pellets) | 3 |
| Cetyl Alcohol | (Lanette 16) | 2.5 |
| Cyclomethicone | (Belsil CM 040) | 0.5 |
| Dimethicone | (Belsil DM 350) | 0.5 |
| Myristil Myristate | (Cetiol MM) | 0.3 |
| Alga extract | (Vegetol Algues) | 0.3 |
| Citronellyl Methylcrotonate | (Sinodor) | 0.3 |
| Allantoin | | 0.2 |
| Xanthan Gum | (Kezan ST) | 0.25 |
| BHT | | 0.15 |
| Ethyl paraben | | 0.6 |
| Methyl paraben | | |
| Phenoxyethanol | | |
| Propyl paraben | | |
| Perfume | | 0.5 |
| Water | | 53.4 |

### Example 9

Day- and night care sets

Day care composition according to Example 2 and night care composition according to Example 2+0.6 or 1.25% Pentavitin.

### Example 10

Further, greasy ointments which may be used as night care composition

| Ingredient | (Trade name) | wt.-% |
|---|---|---|
| Isopropylmyristate | (IPM) | 32.50 |
| Ethylhexyl Hydroxystearate, | (Clearwax) | 6.00 |
| Triethylhexyl Trimellitate, | | |
| C30-45 Olefin | | |
| Cetyl Ricinolate | (Tegosoft CR) | 2.00 |
| Helianthus Annuus | (Sunflower oil) | 20.00 |
| Prunus Dulcis | (Almond oil) | 16.00 |
| Citronellyl Methylcrotonate | (Sinodor) | 0.50 |
| Tocopheryl Acetate | | 3.00 |
| Cetearyl Isononanoate | (Tegosoft CI) | 20.00 |

### Example 11

Further suitable ointments which may be used as night care compositions with 10% urea, 1% Pentavitin und 0.2% sodium hyaluronate

| Ingredient | (Trade name) | wt.-% |
|---|---|---|
| Urea | | 10.00 |
| Glycerine | | 4.00 |
| Glyceryl Stearate | (Tegin M Pellets) | 5.00 |
| Isopropyl Palmitate | (Tegosoft P) | 3.00 |
| Octyldodecanol | (Tegosoft G 20) | 2.50 |
| Cera Alba | (Cera Alba) | 1.50 |
| Cetearyl Isononanoate | (Tegosoft CI) | 2.00 |
| Cetyl Alcohol | (Tego Alkanol 16) | 2.00 |
| Cyclomethicone | (Abil Wax 9840) | 0.50 |
| Dimethicone | (Belsil DM 350) | 0.30 |

| | | |
|---|---|---|
| Isopropyl Myristate | (Tegosoft M) | 6.00 |
| Citronellyl | (Sinodor) | 0.40 |
| Methylcrotonate | | |
| Sodium Hyaluronate | (Hya Care) | 0.20 |
| Sorbitol | (Karion F) | 1.50 |
| Carbohydrate blend | (Pentavitin) | 1.00 |
| Sodium Levulinate, | (Dermosoft 1388) | 3.00 |
| Sodium Anisate | | |
| Citric acid | | 0.10 |
| Tocopheryl Acetate | | 2.00 |
| Water | | 54.36 |

pH-valve adjusted to 5. -5. with citric acid.

### Example 12

Visual Stability Test

The formulations are stored for two weeks at about 11° C. or at about 35° C. and are then sprayed on a plain surface. The stability of the sprayed foams is evaluated visually on the basis of a evaluation scale of 1 to 4, where 1 is the poorest stability and 4 is the best stability.

Examples 1(a-e) are based on the composition according to Example 2 (5.5% urea), Example 2(a-e) are based on the composition according to Example 3 (11% urea) and Example 3(a-e) are based on the composition according to Example 2 or 3, wherein 16.5% urea is comprised. Within the respective formulations a-e, the base and the amount of hyaluronic acid is varied and the post-storage stability is tested.

| | | | | Hyaluronic | | Evaluation of post- | |
|---|---|---|---|---|---|---|---|
| Base | | | | Acid | | storage stability at: | |
| Example | AMP | NaOH | KOH | 0.1% | 0.2% | 11° C. | 35° C. |
| 1a | x | | | | | 1 | 1 |
| 1b | | x | | | | 1 | 1 |
| 1c | | | x | | | 2 | 2 |
| 1d | | | x | x | | 2 | 4 |
| 1e | | | x | | x | 3 | 4 |
| 2a | x | | | | | 1 | 1 |
| 2b | | x | | | | 1 | 1 |
| 2c | | | x | | | 2 | 2 |
| 2d | | | x | x | | 3 | 4 |
| 2e | | | x | | x | 3 | 4 |
| 3a | x | | | | | 1 | 1 |
| 3b | | x | | | | 1 | 1 |
| 3c | | | x | | | 3 | 3 |
| 3d | | | x | x | | 1 | 4 |
| 3e | | | x | | x | 3 | 4 |

It can be seen that the Examples wherein KOH was used were more stable than those where AMP or NaOH were used. Further, it is shown that the use of hyaluronic acid effects an increase in stability and that by the application 0.2 wt-% instead of 0.1 wt-% a further increase in stability is achieved. The best results are obtained by application of KOH as a base and 0.2% hyaluronic acid.

1. Formulation for a foam skin cream comprising an aqueous emulsion and a propellant gas,
in that the emulsion comprising urea and hyaluronic acid, at least one free fatty acid and at least two emulsifiers, wherein the hyaluronic acid has a molecular weight of at least 1×10⁵ Da.

2. The formulation according to claim 1, wherein the hyaluronic acid has a molecular weight of at least 5×10⁵ Da.

3. The formulation according to claim 1, wherein the hyaluronic acid has a molecular weight of at least 1×10⁶ Da.

4. The formulation according to claim 1, wherein the emulsion comprises
3-30 wt-% urea and
0.01-5 wt-% hyaluronic acid.

5. The formulation according to claim 1, wherein the emulsion comprises
5.5-25 wt-% urea and
0.01-0.9 wt-% hyaluronic acid.

6. The formulation according to claim 5, wherein the emulsion comprises about 11 wt-% urea and about 0.2 wt-% hyaluronic acid.

7. The formulation comprising an aqueous emulsion and a propellant gas for a foam skin cream,
the emulsion comprising urea and a carbohydrate blend, which is obtained from naturally available carbohydrates, and
at least one free fatty acid and at least two emulsifiers.

8. The formulation according to claim 7, wherein the carbohydrate blend corresponds in its composition to the carbohydrates contained in human skin.

9. The formulation according to claim 8, wherein the carbohydrate blend is applied in form of an aqueous solution with 50-55 wt-% dry residue.

10. The formulation according to claim 9, wherein the emulsion comprises
3-30 wt-% urea and
0.01-5 wt-% of an aqueous solution.

11. The formulation according to claim 9, wherein the emulsion comprises
5.5-25 wt-% urea and
0.01-0.9 wt-% of an aqueous solution.

12. The formulation according to claim 11, wherein the emulsion comprises about 11 wt-% urea and about 0.6 wt-% of an aqueous solution.

13. The Fformulation according to claim 11, wherein the emulsion comprises about 11 wt-% urea and about 1.25 wt-% of the aqueous solution.

14. The formulation according to claim 1, wherein the emulsion comprises urea, hyaluronic acid and a carbohydrate blend, which is obtained from naturally available carbohydrates and which in its composition corresponds to the carbohydrates contained in human skin.

15. The formulation according to claim 14, wherein the emulsion comprises
5.5 to 25 wt-% urea,
0.01 to 0.9 wt-% hyaluronic acid
0.01 to 2 wt-% of the carbohydrate blend, which is in the form of an aqueous solution with about 50-55 wt-% dry residue.

16. The formulation according to claim 15, wherein the emulsion comprises
5.5 to 25 wt-% urea,
about 0.2 wt-% hyaluronic acid
about 0.6 wt-% or about 1.25 wt-% of the carbohydrate blend.

17. The formulation comprising an emulsion and a propellant gas for a foam skin cream, the emulsion comprising at least one free fatty acid and at least two emulsifiers and the pH-value has been adjusted by potassium hydroxide to 5.5 to 8.

18. The formulation according to claim 17, wherein the emulsion has been adjusted to a pH-value of 6.5 to 7.5.

19. The formulation according to claim 17, further comprising urea and hyaluronic acid according to claim 1.

20. The formulation according to claim 17, further comprising urea and a carbohydrate blend according to claim 7.

21. The formulation according to claim 17, further comprising urea, a carbohydrate blend and hyaluronic acids according to claim 14.

22. The formulation according to claim 19, wherein the emulsion comprises
5.5 to 25 wt-% urea,
about 0.2 wt-% hyaluronic acid and
optionally about 0.6 wt-% or 1.25 wt-% of a carbohydrate blend in the form of an aqueous solution with about 50-55 wt-% dry residue and wherein the pH-value is adjusted by potassium hydroxide to 6.5 to 7.5.

23. The formulation according to claim 1, wherein the at least one fatty acid is selected from C₁₂-C₂₂ fatty acids.

24. The formulation according to claim 23, wherein the at least one free fatty acid is selected from natural C₁₂-C₂₀-fatty acids in particular is selected from stearic acid, palmitic acid and myristic acid.

25. The formulation according to claim 1, wherein the emulsion further comprises unsaturated and/or polyunsaturated fatty acids.

26. The formulation according to claim 25, wherein the emulsion comprises about 1 wt-% evening primrose oil.

27. The formulation according to claim 17, wherein the emulsion comprises about 1 wt-% evening primrose oil and about 16.5 wt-% urea.

28. The formulation according to claim 1, wherein the at least two emulsifiers are selected on the one hand from the group of non-ionic emulsifiers, in particular those on the basis of fatty alcohols and on the basis of fatty acid partial esters and on the other hand, are selected from the group of anionic emulsifiers, in particular, sarcosinates, such as lauryl-, lauroyl- or cetyl sarcosinate.

29. The formulation according to claim 1, wherein the emulsion comprises a further co-emulsifier, selected from the group consisting of lipophilic emulsifiers, such as triceteareth-4-phosphate, Oleth-3-phosphate as well as lipophilic emulsifiers on the basis of low-ethoxylated fatty alcohols.

30. The formulation according to claim 1, wherein the emulsion further comprises at least one refatting substance, selected from the group consisting of decyloleate, isohexadecane, stearic acid glycol esters, coconut fatty acid ethanol amide, corn oil, peanut oil, almond oil, sesame oil, olive oil, jojoba oil, soya bean oil, wool wax alcohols, paraffin, medium-chain triglycerides, oleic acid oleyl esters, white petrolatum, macrogol-glycerol hydroxystearate, hydrogenated castor oil, castor oil from Ricinus communis, avocado oil, wheat germ oil, evening primrose oil, shea butter, palmitic acid isopropyl esters, cetyl palmitate, myristic acid myristyl esters und octyldodecanol.

31. The formulation according to claim 1, wherein the emulsion is paraffin-free.

32. The formulation according to claim 1, wherein the emulsion further comprises at least a further component selected from the group consisting of moisturisers, preservatives, soothing substances, skin care agents, skin active vitamins and silicone containing substances such as dimethicone.

33. The formulation according to claim 32, wherein the emulsion comprises 0.01-2%, preferably 0.1% portulaca.

34. The formulation according to claim 1, wherein the pH-value is from 5.5-8.0.

35. The formulation according to claim 1, wherein the emulsion comprises
1-10 wt-% fatty acids,
5.5-25 wt-% urea,
4-15 wt-% emulsifiers,
0.4-2.5 wt-% co-emulsifiers,
1-10 wt-% moisturisers,
0.05-1 wt-% skin care agents, and
0.5-6 wt-% refatting substances and
0.01-0.9 wt-% hyaluronic acid and/or 0.01-2 wt-% of the carbohydrate blend.

36. The formulation according to claim 1, obtainable by
preparing of a Phase I by melting at 70° C.-80° C. a mixture, comprising at least one fatty acid, and at least one non-ionic emulsifier and at least one co-emulsifier,
metering to a Phase II with stirring which is tempered to 70°-80° C., comprising water, optionally urea, hyaluronic acid and/or the carbohydrate blend, at least one anionic emulsifier to prepare a homogenous mixture of Phase I and Phase II,
cooling the mixture to a temperature from 30° C. to 40° C. with stirring and
optionally adjusting the pH and filling of the resulting mixture in a suitable dosage form with addition of a propellant gas.

37. Foam skin cream, obtainable by spraying the formulation according to claim 1.

38. Day-and-night skin care set, comprising a day skin care composition and a night skin care composition, the day skin care composition is a foam skin cream, comprising at least one free fatty acid and at least two emulsifiers, and the night skin care composition comprises an additional active agent or a higher wt-% content of the active agent or a different active agent as the day skin care composition, whereby the active agent is selected from the group consisting of lipid substances and hydrating substances and mixtures thereof.

39. The day-and-night skin care set according to claim 38, wherein the lipid substances comprise saturated and unsaturated fatty acids as well as the group of refatting substances according to claim 30 and the hydrating substances, urea, high molecular hyaluronic acid, carbohydrate blends, obtainable from naturally available carbohydrates and which correspond in their composition to carbohydrates contained in human skin, and sorbitol.

40. The day-and-night skin care set according to claim 38, wherein the night care composition comprises an additional active agent, namely the carbohydrate blend in the form of an aqueous solution with about 50-55 wt-% dry residue.

41. The day-and-night skin care set according to claim 38, wherein the day skin care composition is a formulation according to claim 1 and the night skin care composition is a formulation according to claim 14.

42. The day-and-night skin care set according to claim 38, wherein the night skin care composition is an ointment.

43. The day-and-night skin care set according to claim 38, wherein the day care composition is a formulation according to claim 26.

44. (canceled)

45. (canceled)

46. Method for treatment of dermatosis, in which a formulation according to claim 1 is applied to the skin area to be treated.

47. Method for treatment of dermatosis, in which on getting up a day skin care composition for day care is applied and prior to going to bed a night skin care composition is applied for night care according to claim 38 is applied to the skin to be treated.

48. Use or method according to claim 46 in which dermatosis comprises “dry skin conditions”, chronic dermatosis, atopic dermatitis, the allergic contact eczema, the irritative contact eczema, psoriasis and the diabetic foot syndrome.

49. The formulation according to claim 2, wherein the emulsion comprises
3-30 wt-% urea and
0.01-5 wt-% hyaluronic acid.

50. The formulation according to claim 2, wherein the emulsion comprises
5.5-25 wt-% urea and
0.01-0.9 wt-% hyaluronic acid.

51. The formulation according to claim 7, wherein the emulsion comprises urea, hyaluronic acid and a carbohydrate blend, which is obtained from naturally available carbohydrates and which in its composition corresponds to the carbohydrates contained in human skin.

52. The formulation according to claim 18, further comprising urea and hyaluronic acid according to claim 1.
